(19) United States
(12) Patent Application Publication
(10) Pub. No.: US 2002/0168321 A1
(43) Pub. Date: Nov. 14, 2002

(54) ADMINISTRABLE MRI COMPOSITIONS FOR ENHANCING THE CONTRAST BETWEEN REGIONS IN ORGANS

(76) Inventors: HERVE TOURNIER, VAILLERY (FR); ROLAND HYACINTHE, MESSERY (FR)

Correspondence Address: ARTHUR R CRAWFORD, NIXON & VANDERHYE PC, 1100 NORTH NORTH GLEBE ROAD, 8TH FLOOR, ARLINGTON, VA, 222014714

(21) Appl. No.: 09/370,893

(22) Filed: Aug. 10, 1999

(30) Foreign Application Priority Data
Aug. 10, 1998 (EP) 98810766.0

(51) Int. Cl. A61B 5/055; A61K 33/26; A61K 31/28; A61B 5/05

(52) U.S. Cl. 424/9.32; 424/9.3; 424/9.322; 424/9.34; 424/9.35; 424/9.36; 424/9.363; 424/9.364; 424/9.365; 424/646; 424/648; 514/492; 514/505; 514/836; 436/173; 600/420

(57) ABSTRACT

The invention relates to injectable compositions for increasing the image contrast between specific organ areas in the magnetic resonance imaging (MRI) of human and animal patients. This enables better differentiation of neighboring tissue portions. Methods of the preparation of such compositions and their use in the MRI of living subjects are also disclosed.

### TECHNICAL FIELD

The present invention relates to injectable compositions for increasing the image contrast between specific organ areas in the magnetic resonance imaging (MRI) of human and animal patients. This enables better differentiation of neighboring tissue portions. The invention also covers the preparation of such compositions and their use in the MRI of living subjects.

### BACKGROUND ART

MRI enables the direct visualization of internal organs in living beings and is therefore a powerful tool in diagnosis, medical treatment and surgery. MRI techniques are based on subjecting a patient to a steady gradient magnetic field directed to the organs of the body to be investigated. The magnetic field acts on hydrogen nuclei of the water embedding such organs and raises them into statistical orientation in a given direction.

For the measurements, short radio-frequency pulses of given resonance energy (which depends on the proton environment) are applied to the region of interest, said pulses transiently disturbing the protons orientation. Then, between two pulses, the protons drop back (relax) to the initial state in a time dependent fashion, thus giving a characteristic signal which is acquired and electronically processed for imaging. When the computerized signal is displayed pixel-wise on a screen, an image is provided of the organ being investigated. In the image, the visual contrast between various domains thereof depends on the differences in spin relaxation time of the water protons belonging to various portions of said organ.

The spin relaxation time constants are expressed in terms of two mutually perpendicular components, T₁ (longitudinal or spin-lattice component) and T₂ (transverse or spin-spin component). Either T₁ or T₂ can contribute to the definition of the NMR images, depending on the measurement mode and conditions.

Visual contrast between the several areas of the image is enhanced if contrast agents (magnetic species) are present in the water in contact with the organs under imaging. The contrast species of interest are the paramagnetic materials (mainly affecting T₁), which provide a positive (+) visual effect, (i.e. they brighten the displayed site in the image) and the negative (−) ferromagnetic or superparamagnetic ones which mainly affect T₂ responses, i.e. the signals are attenuated and the corresponding image area is darkened. The paramagnetic substances include paramagnetic elements in the ionic or organo-metallic state (e.g. chelates of Fe⁻³, Mn⁺², Gd⁺³ and the like). Superparamagnetic substances preferably include very small (<100-200 nm) magnetic particles, for instance magnetite (Fe₃O₄) or ferrite particles.

Although using separately either positive or negative contrast agents in MRI has proven quite useful, attempts have been made to use both (+) and (−) contrast agents simultaneously.

Thus, for instance, R. Weissleder et al. (Am. Journ. Radiol. 150 (1988), 561-566) have reported MRI experiments involving injecting rats carrying liver tumors with either Gd-DTPA (a) alone (as T₁ (+) contrast agent), or ferrite (b) alone (as T₂ (−) contrast agent), and eventually (c) both the foregoing (+) and (−) contrast agents together. After injection, the Gd-DTPA first distributes evenly into liver extra-cellular space and, after a while, it is preferentially retained within the tumors wherefrom it is ultimately excreted through the kidneys. When imaging under T₁ weighted sequences a few min after injection, the tumors appear bright (brighter than without the contrast agent) relative to the healthy liver. The reason why the Gd-DTPA remains tumors longer than in the normal liver tissue is unclear but may be due to differences in vascularization. Ferrite (considered a typical RES-specific super-paramagnetic contrast agent) is rapidly taken up by hepatic Kupffer cells and, as a result, under T₂ weighted sequences the liver parenchyma tissue appear darker than the tumors. Actually, according to the reference, “T₁-weighted pulse sequences such as SE/500/30 have sufficient T₂ contrast dependence to create a T₂-weighted ferrite-enhanced image”.

Then, liver imaging with co-administration of both paramagnetic contrast agent (a) and ferrite (b) provided results combining the advantages of the two contrast agents. For instance, ten min after administration of both, the tumor signal intensity was increased by the Gd-DTPA (predominant T₁ shortening) and the liver signal intensity was concomitantly decreased by ferrite (predominant T₂ shortening). As a result, C/N (contrast to noise) values were greater than with either (a) or (b) alone. This technique does not however enable to distinguish vascularization over cellular tissue.

U.S. Pat. No. 5,128,121 also defines ferromagnetic and superparamagnetic MRI contrast additives as negative (−) contrast agents whose effect is to reduce the T₂ proton spin component. The same document however defines the paramagnetic contrast agents as either positive (+) (especially at low concentrations where the effect on T₁ is predominant), or (−) in cases the effect on T₂ predominates (situation pertaining to special conditions involving higher concentrations and other factors).

According to this document visualization in tissue or organ MRI can be enhanced by administration of tissue-specific positive and negative contrast agents, or body duct-specific positive and negative contrast agents. Tissue- or duct-specificity refers to the fact that following administration the agent does not distribute widely but substantially remains or concentrates within a specific tissue or body duct or cavity during imaging time. An example of a duct specific contrast agent is a “blood pool” agent that, after injection, is preferentially retained within the cardiovascular system. This contrasts with, for instance, the behavior of Gd-DTPA that rapidly distributes extra-cellularly in a body volume about 5 times larger than the circulation. Examples of agents with tissue-specificity are the tissue targeting agents like the hepatobiliary paramagnetic phenylimidodiacetates of EP-A-0 165 728 and the RES targeting magnetite-carbohydrate particles of WO-A-85/04330. The reference also indicates that for best contrast results the two agents should distribute within different body volumes; hence it also discloses contrast compositions containing at least one negative and one positive tissue-specific or duct-specific contrast agents.

With regard to the combined effect of positive and negative contrast agents, it should be incidentally reminded that although each acts mainly on one of the two mutually perpendicular proton relaxation components, they, although to a lesser extent, also have an influence on the other component. Thus the overall effect of the mixture is in fact equal to the effect of both T₁ and T₂. In other words, the T₁ of a mixture of a positive and a negative contrast agent is shorter than the T₁ of the positive contrast agent alone. The same considerations apply to the T₂ factor.

The following documents provide general embodiments of magnetite particles usable as negative contrast agent (disclosures of which are incorporated herein by reference).

EP-A-0 186 616 discloses the preparation of suspensions of super-paramagnetic particles by alcalinization of metal salt solutions (for instances solutions of Fe⁺² and Fe⁺³), then coating with polymers such as polysaccharides or proteins. The particles can be smaller than 50 nm.

U.S. Pat. No. 4,101,435 discloses the preparation of dextran-coated magnetic particles. For instance, there are prepared an aqueous suspension of magnetite particles and an aqueous solution of purified dextran. The solutions are admixed together and coating becomes effective under reflux. After cooling, the particles are collected, resuspended in water, purified by dialysis and finally recovered by lyophilisation.

WO-A-85/04330 discloses suspension of magnetite particles that may be free or coated with bio-tolerable polymers, e.g. cellulose derivatives or BSA.

EP-A-0 186 616 discloses the preparation of magnetite microparticles (˜200 nm) “conjugated” with HSA.

U.S. Pat. No. 4,267,234 discloses the preparation of magnetite particles (Ferrofluid) coated with polymerized glutaraldehyde in aqueous emulsion. By this method, 100 nm magnetic beads are obtained. The latter can be isolated with a magnet, washed and finally resuspended in buffer.

Coated or uncoated magnetite particles usable as the short lasting contrast component of this invention are also described in WO-A-83/03426.

WO-A-88/00060, discloses superparamagnetic particles of 5-50 nm which are coated with polymers or surfactants to prevent subsequent particle agglomeration. The polymers include polysaccharides like dextran, proteins and polypeptides. For instance, for preparing the coated superparamagnetic particles, a 1:2 (molar ratio) mixture of FeCl₂/FeCl₃ is brought to basic pH with ammonia and the precipitated powder is dispersed by sonication, oxidized and the particles are coated by adding the coating material under dispersion.

Similar techniques are disclosed in WO-A-88/06632 and WO-A-85/04330 disclosure of which is incorporated herein by reference.

A long lasting negative contrast component is exemplified by super-paramagnetic magnetite particles protected against removal by the RES. These are disclosed for instance in WO-A-94/04197.

Although the achievements of the prior art have merit, they are not designed to contrast the circulation relative to its surroundings and, as a consequence, they suffer from the drawback of not enabling to clearly observe vascularity against the cellular background of organs. This is important since the visualization of the vascular network helps differentiating normally vascularized tissue from pathologic ones like hypoxia tissue (for instance in tumors). The present invention markedly improves the situation in this regard.

### SUMMARY OF THE INVENTION

A first object of the invention is to provide administrable dual MRI contrast enhancing compositions containing as key components, at least (a) one positive paramagnetic metal chelate contrast agent and at least (b) one negative ferromagnetic or superparamagnetic contrast agent. These compositions distinguish the prior art by the properties of the said components toward the cell membrane barrier. Actually, either one of (a) and (b) predominantly internalizes tissue, whereas the remaining one is predominantly retained in the circulation, this being for a time sufficient to provide sharp MRI images of the circulation in said tissue. Typically, either one of (a) and (b) is predominantly intra-vascular while the other one is predominantly extra-vascular or is rapidly removed from the circulation by macrophages. Then, after removal from circulation it internalizes neighboring tissue. The transfer from vessels to tissues is effected by RES mediated phagocytosis. Alternatively, an extra-vascular compound may cross the vessel walls and distribute randomly extracellularly.

Another object of the invention is to provide a dual blood pool contrast medium comprising a positive MRI contrast agent (a) mainly shortening the T₁ relaxation response and a negative contrast agent (b) mainly shortening the T₂ relaxation response, both relaxation effects of (a) and (b) being controllable at will.

Another object of the invention is to provide a method of making the MRI contrast compositions fulfilling the above requirements, that is, making MRI contrast compositions or media simultaneously containing intra- and extra-vascular contrast agents. One particular object is to provide ingredients and conditions such that both key components (a) and (b) will exist simultaneously in adequate populations in one solution or suspension within a pharmaceutically acceptable carrier liquid, the degree of vascular retention (and disappearance) with time of the (a) and (b) components after administration being controllable at will.

A further object of the invention is the use of the present compositions for enhancing the image contrast between contiguous areas in the MRI of organs of patients. Under such use conditions, the present composition is administered, preferably by intravenous (IV) injection, to a subject and MR visualization of selected areas is effected at intervals, the degree of contrast between selected domains under investigations being measured according to usual means. The results, and particularly the change with time of the vessel to tissue signal ratio (i.e. the contrast variation with time after administration), can thereafter be monitored and interpreted by trained personnel to provide highly diagnostically useful information.

Yet another object of the invention is a kit comprising the two components one or both of which may be in a powder form. If one of the components is in a powder form the kit may further include a physiologically acceptable carrier liquid.

Still further object of the invention is a method of making the dual component MRI medium.

### BRIEF DESCRIPTION OF THE DRAWINGS

FIG. 1 refers to a photographic MRI display showing organ vascularization after injection into a rabbit of a contrast medium according to the invention.

FIG. 2 refers to a photographic MRI display showing organ vascularization after injection into a rabbit of a control, i.e. a negative contrast agent alone.

FIG. 3 refers to a photographic MRI display showing organ vascularization after injection into a rabbit of a contrast medium according to the invention.

FIG. 4 refers to a photographic MRI display showing organ vascularization after injection into a rabbit of a control, i.e. a positive contrast agent alone

### DETAILED DESCRIPTION OF THE INVENTION

If in a first general embodiment of the present invention, the blood pool imaging agent is identified as the positive (+) contrast agent, it is preferably constituted by micellar particles of paramagnetic metal ions complexed with a chelating agent having a lipophilic moiety, this being in association with one or more amphipatic organic compounds. This kind of material is disclosed in WO-A-97/00087 incorporated herein by reference. The chelating agent comprises a polyamino-polycarboxylate backbone carrying at least one lipophilic substituent; for instance one carboxylate function thereof is esterified with a fatty alcohol or amidated with a saturated or unsaturated long chain amine. Preferably, the lipophilic moiety of the paramagnetic metal complex has a C₁ to C₂₄ alkyl or alkylene group or a substituted or unsubstituted benzyl- or phenyl-alkyl group.

In this embodiment, complexes of paramagnetic metal ions with lipophilic chelating agents are referred to as the positive (+) paramagnetic imaging components (a). These positive imaging components of the invention are actually more or less tight associations of imaging contrast agents, non-ionic surfactants and optionally phospholipids, and they preferably are in the form of stable mixed micelles suspended in a suitable carrier liquid. The mixed micelles are constituted by the conjugation or association of a lipophilic metal chelate with a non-ionic surfactant and optionally an amphipatic compound. The term association or conjugation means that the components of the micelles may be in the form of adducts or admixtures of two or more substances having mutual affinity for each other. Or the association may be due to one or more bonds e.g. H-bonds between the constituents, whereby a chelatant molecule with simultaneous lipophilic and hydrophilic properties will ensue in a given desirable equilibrium (appropriate hydrophilic/lipophilic balance). Hence, the imaging component (a) may consist of a mixture of an amphiphile and a paramagnetic chelate species bearing a function possessing affinity for the amphiphile. Such a structure resists ready metabolization by the macrophages and is particularly long lasting in the circulation.

Clearly, the presence of one or more non-ionic surfactants is essential since the non-ionic surfactant causes the principal constituents i.e. the paramagnetic metal chelate having a lipophilic function, the phospholipid and the surfactant to form mixed micelles. By rendering the principal constituents of the composition micellar, the properties of the active contrast component change and unexpectedly long lasting imaging properties are obtained. The size of the micelles is found to vary between 10 and 800 nm, however, it appears that the most effective results are obtained when the size is preferably between 30 and 500 nm. Dispersed in a suitable aqueous carrier liquid, the mixed micelles form very stable colloidal dispersions of custom controlled stability, i.e. the micelles will resist agglomeration, aggregation and eventual collapse for a period of time to be controlled by the kind and relative proportions of the chelate, the surfactant and the phospholipid.

In the positive component (a) of this embodiment of the invention, the polyaminopolycarboxylate chelating agent is provided with a hydrophobic group (for instance, an esterified fatty alcohol chain) which readily couples or intertwines (presumably due to Van der Waals forces) with the hydrophobic part of a non-ionic surfactant and optionally with the fatty acid residues of the phospholipid. The non-ionic surfactant (preferably a polyoxyethylene-polyoxypropylene block copolymer) presumably provides the additional hydrophilic/lipophilic balance parameters, which enable the complex system to exist in the form of mixed micelles dispersed in a carrier liquid.

To be consistent in the afore discussed embodiment, the negative contrast agent (b) admixed in various proportions with the positive contrast agent (a) in the contrast enhancing medium of the invention is preferably constituted by colloidal superparamagnetic magnetite particles of submicronic size. In the presently described embodiment, the magnetite particles (b) co-injected with the micelles (a) will stay suspended in the blood for only a relatively short time (which can be controlled at will as discussed below); then they become internalized and immobilized by the RES, thus outlining the surrounding tissues. When this is effective, MRI measurements are undertaken under T₁ or T₂ weighted conditions depending on the needs. It is one of the strong advantages of the invention that both the T₁ and T₂ relaxation factors can intervene in the provision of MRI images of enhanced contrast between the blood vessels and the surrounding tissues. In most cases images taken under either T₁ or T₂ sequences are effective. When under T₁, the vessels will appear very bright against relatively unchanged surroundings, whereas under T₂ the vessels will appear about normal against a much darker background. In fact, there is only a general shift of the whole image toward the darker side (or the brighter one depending on whether the change is from T₂ to T₁, or the other way round), but the light output difference between the darker and brighter areas remains about the same. Compounding the images will then reveal the full potential and benefits of the invention.

The foregoing may possibly be better understood by reference to some typically exemplified cases described hereafter.

Indeed, taking into account that the main effect of the magnetite particles is to lower the T₂ factor (and also the T₁, but to a lower extent), i.e. it provides a negative (−) darkening effect, whereas the main effect of the paramagnetic micelles is to lower the T₁, i.e. to brighten the addressed area, the blood vessels will actually appear brighter than normal against a tissue background (e.g. the liver) darker than usual. Hence, comparison between the following three possible systems will give the interesting results below:

1) Injection of blood-pool micelles (a) alone

A). T₁ weighted sequences: inside very white, outside gray (no effect).

B). T₂ weighted sequences: no particular effect.

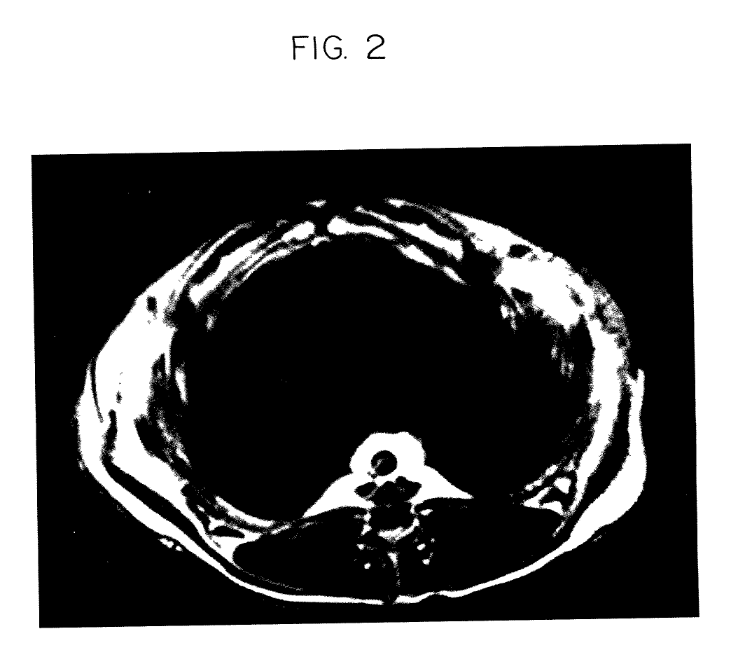

2) Magnetite particles (b) alone (see FIG. 2)

A). T₁ weighted sequences: no effect

B). T₂ weighted sequences: inside gray (no particular effect), outside blacker.

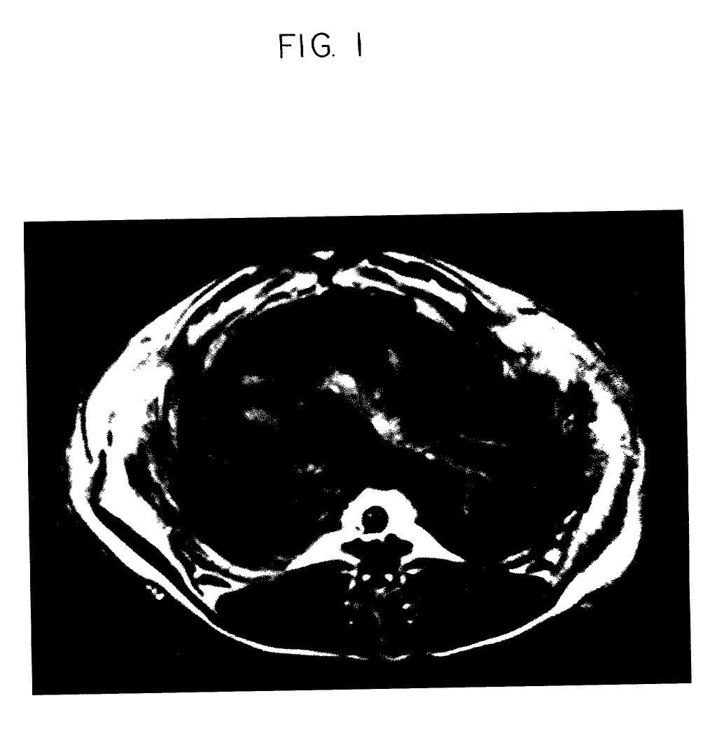

3) Injection of the present dual contrast agent (magnetite (b)+Micelles (a), see FIG. 1)

A). T₁ weighted sequences: inside very white, outside darker than in case 1A.

B). T₂ weighted sequences: outside very black, inside whiter than in case 2B.

The magnetite particles to be profitably used in this invention are many. The particle size is preferably in the 50 to 300 nm range and re-aggregation on standing to coarser sizes can be prevented by the presence of additives like sugars or polyols; or the particles can be coated with a layer of protective material. By properly selecting the kind of protective material and thickness of the coating layer, one can control the time of residence of the particles in the blood after injection. So many kinds of magnetite particles of the prior art are useful in the invention. Some of the pertinent prior art concerning said magnetic particles has been reviewed in the prior art portion of this disclosure and is incorporated herein by reference.

Of course, in a contrasting second general embodiment of the present invention, the negative contrast component (b) can have blood-pool properties, whereas the positive contrast component (a) is rapidly removed from circulation, e.g. extra-vascularly or otherwise.

A convenient long lasting negative contrast component (b) is exemplified by super-paramagnetic magnetite particles protected against removal by the RES. These are disclosed for instance in WO-A-94/04197 disclosure of which is incorporated herein by reference.

The negative blood pool contrast agents usable in said second embodiment of the present invention comprise iron oxide particles stabilized by a three dimensional shell layer containing molecules of an amphipatic compound and a non-ionic surfactant. This shell makes the particles invisible to opsonin rendering them macrophage resistant.

The amphipatic compound has a hydrophilic negatively charged phosphorus containing head moiety bonded to a hydrophobic tail moiety and is present in divided form due to the influence of the non-ionic surfactant in the three dimensional shell layer and to the hydrophilic phosphorus containing (preferably phosphoryl) head moiety of the amphiphile which bears at least two negative charges.

The three dimensional shell is formed from molecules of the amphipatic compound whose negative phosphoryl head moieties are pointing towards the iron oxide core and the hydrophobic tail moieties protrude outwardly therefrom forming an urchin-like structure. The urchin-like structure serves as a base for building the three dimensional shell by anchoring thereto the non-ionic surfactant. When the amphipatic compound is a mono-alkyl or -alkenyl phosphoric acid ester or a glycerophospholipid, the outer layer comprises a non-ionic surfactant whose hydrophobic moieties are interlaced or intertwined with the alkyl or alkenyl chain of the ester or glycerophospholipid; this arrangement further stabilizes the structure. In either case, the natural ability of the non-ionic surfactant to cause micellization of these compounds is effective. The non-ionic surfactant is preferably a block POE-POP polymer, a polyoxyethylene fatty acid ester, a polyoxyethylene sorbitan fatty acid ester, an n-alkylglucopyranoside, or an n-alkyl maltotrioside. The phosphorus compound is preferably a phospholipid selected from phosphatidic acids, phosphatidylcholine, phosphatidylethanolamine, phosphatidylserine, phosphatidyl-glycerol, phosphatidyl-inositol, cardiolipin, sphingomyelin or a mono-phosphate ester of a substituted or partially substituted glycerol, at least one functional group of said glycerol being esterified by saturated or unsaturated aliphatic fatty acid, or etherified by saturated or unsaturated alcohol, the other two acidic functions of the phosphoric acid being either free or salified with alkali, earth-alkali metals or organic amines (ammonium compounds).

In the present embodiment involving a negative contrast blood-pool component, the positive other component (a) will have to readily quit the circulation and intra- or extracellularly internalizes neighboring areas surrounding the blood vessels. Neat chelates of paramagnetic metals commonly used in the field of MRI as general or hepatobiliary contrast agents are convenient. These include for instance chelate moieties such as EDTA, DTPA, BOPTA, DOTA, DO3A and/or their derivatives, while the paramagnetic metal may be selected from Gd(III), Mn(II), Cr(III), Cu(II), Fe (III), Pr(III), Nd(III) Sm(III), Tb(III), Yt(III) Dy(III), Ho(III) and Er(III).

For the preparation of the dual components, positive/negative contrast media of the invention, (one component of which remains in the blood and the other goes readily into the surroundings), one can admix together the priorly made components (a) and (b) in a suitable physiologically acceptable injectable liquid carrier. Alternatively, one can prepare the two components in succession in situ, i.e. directly in the final carrier liquid, usually a buffered aqueous solution.

For instance, to exemplify the second of the above discussed embodiments, one can disperse a freshly prepared portion of magnetite particles obtained by NH₄OH precipitation of a FeCl/FeCl₃ solution in a suitable buffer and add suitable proportions of DPPA (dipalmitoylphosphatidic acid) and Pluronic F-108 (a polyoxyethylene-polyoxypropylene block polymer) in order to provide stealth properties to the magnetite (the negative contrast agent). Thereafter, one admixes a portion of a typical hepatobiliary contrast agent, for instance Gd-BOPTA so that the two active components are in a weight ratio of from 10:1 to 1:10, ratios of 2:1 to 1:2 being preferred. Before administration to experimental animals, an aliquot of the above preparation is diluted to a desired concentration, generally from about 0.001 to 0.1 molar, and injected intravenously in the circulation. Then after a period of time, the organs of interest of the animal are imaged under regular MRI conditions, a series of successive images being taken at intervals under T₁ and/or T₂ weighted sequences. The responses gathered from the images thus obtained enable to provide enhanced contrast data of the circulation against the background tissues in various organs, for instance the brain, the liver and the spleen.

In the case of the earlier embodiment discussed in this specification (i.e. when the positive component (a) remains in the blood whereas the negative component (b) is removed and becomes immobilized), one may proceed similarly but using contrast components with opposite properties.

For instance one may admix together in a suitable injectable buffer uncoated magnetite particles or magnetite particles coated with a light biodegradable coating like dextran or HSA, and suitably hindered paramagnetic chelates such as the micelles discussed above in connection with document WO-A-97/00087. The amount of coating material used on the magnetite particles may vary from about 0.001 to 1% by weight, the greater the amount of coating material, the longer the particles will stay in the blood. Then the dual component contrast medium thus obtained is tested along the same lines disclosed in regard of the second embodiment.

In view of the fact that one or more components of the dual contrast agent of the invention may be produced and stored in a powder form yet another object of the invention is a kit comprising the active components of the dual contrast medium.

For practically applying the compositions of the invention in the kit form the dried components and the carrier liquid marketed separately are reconstituted by mixing together the kit components to reconstitute the dual contrast medium prior to injection into the circulation of patients.

The dry powders of the kit may be stored under atmosphere of an inert gas while a physiologically acceptable carrier liquid, may further contain isotonic additives and other physiologically acceptable ingredients such as various mineral salts, vitamins, etc.

As already mentioned the reconstituted medium is particularly suitable for use in MRI imaging of organs in human or animal body. These compositions could facilitate MRI angiography, help assessment of myocardial and cerebral ischemia, pulmonary embolism, vascularisation of tumors, tumor perfusion etc.

The following Examples illustrate the invention in more detail.

### EXAMPLE 1

### Dual Contrast Composition Comprising a Positive Remnant Blood Pool Contrast Agent and a Fugitive Negative Contrast Agent

A. Preparation of a contrast composition

In a vessel, there were dissolved in 250 ml of water 3.93 g (14.54 mmol) of FeCl₃.6H₂O and 2.93 g (14.74 mmol) of FeCl₂.4H₂O. An aqueous 25% solution of ammonia was added drop-wise under stirring until the pH was 9.0. A suspension of black magnetite particles formed which was heated to 75° C. for 5 min, after which the suspension was allowed to stand at r.t. whereby the particles settled at the bottom of the vessel. The precipitate was washed by decantation with several 500 ml portions of Tris-glycerol (Tris 1 g/l; glycerol 0.3M). After washing, the particles were re-dispersed in 500 ml of Tris buffer, pH 7.25. The iron concentration in this suspension was 3.01 mg Fe/ml.

To 50 ml of the above dispersion were added successively 6 g of a hydrophobized paramagnetic compound of formula

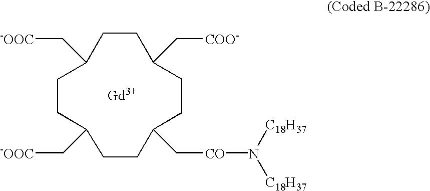

then, 6 g of a polyglycol-modified phosphatidyl ethanolamine (coded DSPE-MPEG 2000) of formula

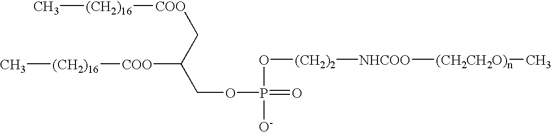

Distearoylphosphatidylethanolamine-methoxypolyethylene glycol 2000

(n=approximately 45) available from SYGENA/GENZYME of Switzerland, then 250 ml of tris-glycerol buffer pH 7.25. (The compound B-22286 had been prepared by amidation of DOTA according to usual means).

Then the solution was sonicated for 15 min with a BRANSON 250 Sonifier, output 60. The temperature which had risen to 70° C. was allowed to return to normal and the dual suspension (magnetite particles+paramagnetic micelles) was filtered on 0.45 μm sterile filter (millipore) and stored in sterilized bottles.

A control composition was prepared as above but omitting the above Gd chelate.

B. Imaging of the liver of the rabbit

The foregoing contrast composition (and the control) were injected to experimental rabbits; the dose was 2 ml/kg, namely 1 mg of Fe and 38 μmol of Gd. Imaging of abdominal transverse sections was carried out using a MR imager from Picker International, Inc. Eclipse 1.5T. The operating conditions were as follows:

Sequence: SE (spin echo); TE=12.0 ms; TR=400 ms; FOV=16.0 mm; Flip angle=90°; 256×256.

The results are clearly visible from the figures.

FIG. 2 refers to an image obtained after injection of the control, i.e. magnetite alone; the blood vessels appear grayish (moderate signal) against the black liver parenchyma (very weak signal).

FIG. 1 refers to an image obtained after injection of the composition according to the invention; the blood vessels appear white (strong signal) against the black liver parenchyma (very weak signal).

If in the present Example, the compound B-22286 is replaced by other remnant paramagnetic chelates as listed below, similar results are obtained.

[10-Hexadecyl-1,4,7,10-tetraazacyclododecane-1,4,7-triacetato(3-)]gadolinium

[10-Octadecyl-1,4,7,10-tetraazacyclododecane-1,4,7-triacetato(3-)]gadolinium

[10-(2-Hydroxyoctadecyl)-1,4,7,10-tetraazacyclo-dodecane-1,4,7-triacetato-(3-)]gadolinium

[10-[2-(Dioctadecylamino)-2-oxoethyl]-1,4,7,10-tetraazacyclododecane-1,4,7-triacetato (3-)]gadolinium

[10-[2-Hydroxy-3-[[[2-(Octadecyloxy)-1-[(octadecyloxy)methyl]ethoxy]-acetyl ]oxy]propyl]-1,4,7,10-tetraazacyclododecane-1,4,7-triacetato(3-)]gadolinium

The preparation of the above compounds is effected by usual means in analogy with that disclosed in Example 4.

### EXAMPLE 2

### Dual Contrast Composition Comprising a Negative Remnant Blood Pool Contrast Agent and a Fugitive Positive Contrast Agent

The following ingredients were successively added to 50 ml of the dispersion of magnetite particles described in Example 1: (a) 4.5 g of DPPA.Na from Sygena (Na salt of dipalmitoylphosphatidic acid), (b) 4.5 g of Synperonic F-108 (a polyoxyethylene-polyoxypropylene block-copolymer from ICI), (c) 9.6 g of Gd-BOPTA, (as the dimeglumine salt), i.e. the complex of gadolinium with the chelate having the following formula

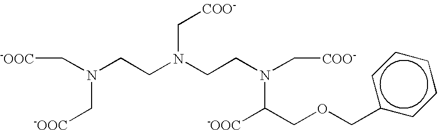

the latter being made neutral with 2 meglumine equivalents, and (d) 250 ml of tris-glycerol buffer, pH 7.25. Then, the mixture was sonicated for 20 min with a Branson Sonifier, output 60, after which the temperature (80° C.) was allowed to drop to normal. The suspension containing 0.5 mg Fe/ml and 30 μmol of Gd/ml was filtered on 0.45 μm membrane and stored in sterilized containers.

A similar control was prepared as above but omitting the magnetite particles.

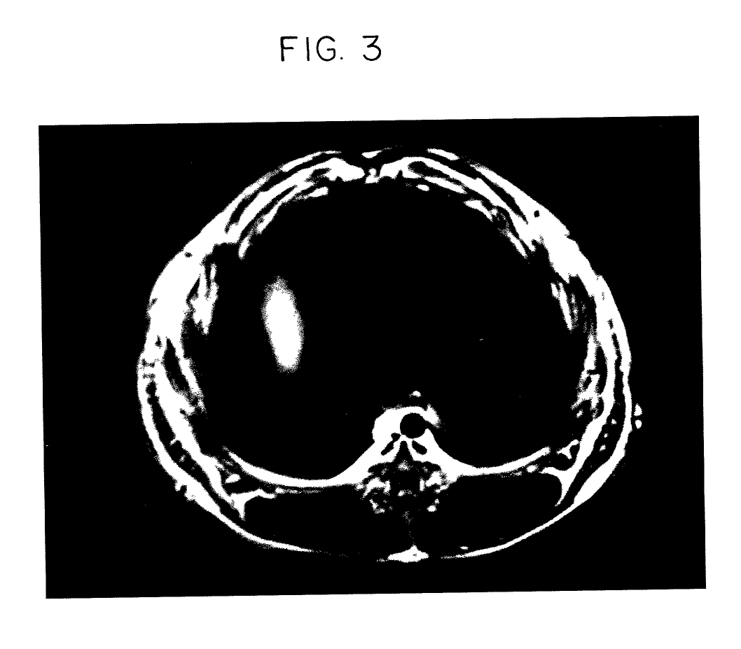

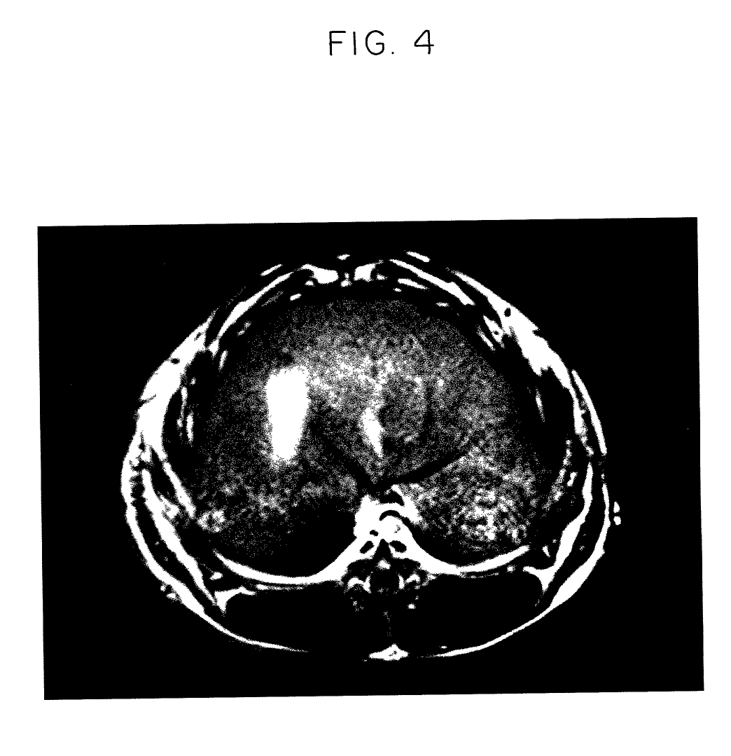

Testing the dual contrast composition of this Example was effected as in Example 1. The results are visible In FIGS. 3 and 4. In the control, the blood vessels (grayish) do not much contrast with parenchyma tissue appearing white; in the case of the composition of the invention, the signal from the blood vessels is inhibited and the latter appear contrastingly black against the white surroundings. Actually, the respective output signals pertaining to FIGS. 3 and 4 provide the opposite effect of that in FIGS. 1 and 2.

### EXAMPLE 3

A suspension of magnetite particles was prepared as described in Example 1, the iron concentration of which was 3.01 mg Fe/ml.

To 5 ml of this suspension were added 600 mg of the following compound (coded B-23146)

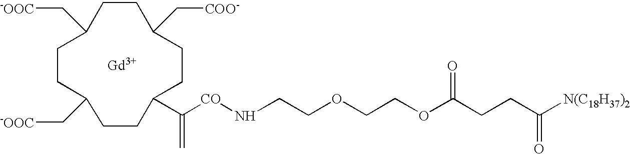

600 mg of DSPE-MPEG 2000 (see Example 1), and 25 ml of tris-glycerol buffer, pH 7.25.

Hence, the concentrations were as follows: 0.5 mg Fe/ml; 20 mg of B-23146/ml i.e. ˜16 μmol of Gd/ml, and 20 mg/ml of PE-PEG. The size of the particles was in the 20-50 nm range.

Relaxivity measurements

Proton spin relaxivities of the foregoing preparations were measured using a Minispec PC-120 (Bruker) apparatus, operating under 0.47 Tesla (20 MHz). EDM 510A (EDM=Experiment Definition Module) was used to measure the spin-lattice relaxation time T₁ by the “inversion recovery” method. EDM 610A was used to measure the spin-spin relaxation time T₂ by the Carr-Purcell-Meiboom-Gill (GPMG) technique. The results are provided in terms of ms (T₁ and T₂).

For preparing the samples to be measured in buffer, 1 ml aliquot of the above-described preparation was diluted to 20 ml with buffer, and a 5 ml portion of the dilution was placed in a Bruker measuring tube. For blood measurements, 0.5 ml of the preparation was diluted with 9.5 ml of rabbit blood.

According to initial results, T₁ was 43.6 ms in buffer and 44.7 ms in blood; T₂ was 8.5 ms in buffer and 8.0 ms in blood.

Injection in rats

One ml of the above preparation was injected into the tail of experimental rats.

Then, after a period of time (see Table below) the animals were sacrificed and the blood and the liver were tested for T₁ and T₂ as above. For measuring the liver, the organ was sliced with water to make 35 g, and then the mass was homogenized using a Polytron mixer head. From this, a 5 g aliquot was taken and placed in a Bruker tube for the measurements. The above operations were performed using 3 rats of about equivalent weight (300-310 g), the intervals of time after injection being 5, 15 and 30 min, respectively. The results in ms are outlined in the Table below. Another rat was used for controlling the initial conditions.

| | |
|---|---|
| Blood | Liver |

| Time (min) | | T₁ | T₂ | T₁ | T₂ |
|---|---|---|---|---|---|
| | 0 | 800 | 200 | | |
| | 5 | 72.5 | 28.9 | 259 | 16.2 |
| | 15 | 79.4 | 57.7 | 262 | 17.6 |
| | 30 | 84.5 | 60.7 | 221 | 16.0 |

The above results show that the effect of injection is considerable on both T₁ and T₂. The time reduction is however greater with regard to T₂ than with regard to T₁ because after 5 min there remains traces of magnetite in the blood. The “non-stealth” magnetites are rapidly taken up by the liver tissue, wherefrom the considerable relaxation time reduction (T₂→16 ms).

After 15 min, the effect on T₁ in the blood is still there because the “stealth” paramagnetic micelles are still in circulation. The effect on T₂ decreases since there is less and less magnetite in the blood. In the liver, the effect of the magnetites on T₂ remains practically the same.

After 30 min there is not much change; the effect of the positive contrast component is still predominant in the blood.

### EXAMPLE 4

### Preparation of Compound [10-[2-(Octadecylamino)-2-Oxoethyl]-1,4,7,10-Tetraaza-Cyclododecane-1,4,7-Triacetato-(3-)]Gadolinium] of Formula

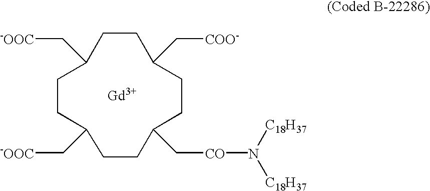

A) 2-Bromo-N-octadecylacetamide (C.A.S. registry number 15491-43-7)

A solution of bromoacetyl bromide (44.4 g; 0.22 mol) in CH₂Cl₂ (50 ml) was added drop-wise in 2.5 h at 20° C. to a mixture of octadecylamine (59.3 g; 0.22 mol) and K₂CO₃ (30.4 g; 0.22 mol) in CH₂Cl₂ (600 ml) and H₂O (600 ml). After 16 h at room temperature the organic layer was separated, washed with H₂O, dried over Na₂SO₄ and evaporated. The crude product was purified by flash chromatography (CH₂Cl₂/MeOH=100/1 (v/v)) to give the desired product (60 g; 0.154 mol). Yield 70%.

GC: 96% (area %); K.F.: <0.1%; ¹H-NMR, ¹³C-NMR and MS spectra were consistent with the structure.

Elemental analysis (%):

| | C | H | N | Br | O |
|---|---|---|---|---|---|
| Calcd. | 61.52 | 10.33 | 3.59 | 20.46 | 4.09 |
| Found | 61.75 | 10.71 | 3.58 | 20.14 | 4.01 |

B) 10-[2-(Octadecylamino)-2-oxoethyl]-1,4,7,10-tetra-azacyclododecane-1, 4,7-triacetic acid

A mixture of 1,4,7,10-tetraazacyclododecane-1,4,7-triacetic acid tris(1,1-dimethylethyl) ester (24 g; 46.6 mmol) and 2-bromo-N-octadecylacetamide (18.2 g; 46.6 mmol) in EtOH (500 ml) was heated to reflux. After 2.5 h the reaction mixture was evaporated, the residue was dissolved in CH₂Cl₂ and CF₃COOH was added. After 15 min the solvent was evaporated and the oily residue dissolved in CF₃COOH. After 16 h at room temperature the solution was evaporated and the oily residue was purified by flash chromatography (CH₂Cl₂/MeOH=3/1 (v/v); then CH₂Cl₂/MeOH/NH₄OH 25% (w/w)=12/4/1 (v/v/v)).

The product was dissolved in H₂O and 6 N HCl, the solution was loaded onto an Amberlite XAD-8 resin column and eluted with a CH₃CN/H₂O gradient. The product elutes with 50% CH₃CN.

The fractions containing the product were evaporated and dried under reduced pressure to give the desired product (12 g; 18 mmol). Yield 39%.

Acidic titer (0.1 N NaOH): 91%; HPLC: 95% (area %): .F.: 8.82%; ¹³C-NMR, MS and IR spectra were consistent with the structure.

Elemental analysis (%):

| | C | H | N | |
|---|---|---|---|---|
| Calcd. | 62.26 | 9.99 | 10.68 | |
| Found | 62.28 | 9.63 | 10.64 | anhydrous |

C) [10-[2-(Octadecylamino)-2-oxoethyl]-1,4,7,10-tetra-azacyclododecane-1,4,7-triacetato(3-)]gadolinium

Gd₂O₃ (1.97 g; 5.4 mmol) was added to a solution of the free ligand from the previous preparation (7.12 g; 9.7 mmol) in H₂O (310 mL) and the resulting suspension was heated at 50° C. for 9.5 h. The reaction mixture was filtered through a Millipore apparatus (HA 0.45 mm filter) and the solution was evaporated to give the title compound (8.6 g; 9.5 mmol). Yield 98%.

HPLC: 98% (area %); K.F.: 9.98%; MS and IR spectra were consistent with the structure.

Elemental analysis (%):

| | C | H | N | Gd | |
|---|---|---|---|---|---|
| Calcd. | 50.41 | 7.71 | 8.64 | 19.41 | |
| Found | 50.52 | 7.78 | 8.65 | 19.32 | anhydrous |

1. An injectable MRI dual component contrast medium for imaging blood vessels in organs, comprising as a dispersion in a physiologically acceptable carrier liquid (a) a positive contrast agent component acting on the T₁ proton relaxation factor, and (b) a negative contrast agent component acting on the T₂ proton relaxation factor, characterized in that one of said components is predominantly an intravascular blood pool contrast agent remaining in the blood vessels for a period of time, while the other one is rapidly removed from the blood thus increasing the contrast between blood and tissue.

2. The composition of claim 1, wherein said predominantly intravascular contrast component (a) is a blood remnant paramagnetic metal chelate compound, and the other contrast component (b) comprises submicronic magnetite particles readily and controllably taken up by the RES.

3. The composition of claim 2, wherein said positive blood pool agent (a) comprises micellar particles of paramagnetic metal ions complexed with a chelating agent having a lipophilic moiety, the complex being associated with one or more amphipatic organic compounds.

4. The composition of claim 2, wherein said magnetite particles of component (b) are coated with a layer of biodegradable material whereby control of the rate of penetration into the surroundings can be effected.

5. The composition of claim 1, wherein said negative contrast component (b) is remnant in the blood, while the positive component (a) readily leaves to penetrate into the surroundings.

6. The composition of claim 5, wherein said negative contrast component (b) comprises particles of magnetite surrounded by spike-oriented chains of an amphipatic phosphorus compound having a phosphoryl head moiety electrostatically bound to the magnetite and a hydrophobic chain tail moiety pointing outwards, and one or more non-ionic surfactants.

7. The composition of claim 6, wherein said one or more surfactants prevent said tail moieties from coalescing together, thus making the particles invisible to opsonin and macrophage resistant.

8. The composition of claim 6, wherein said surfactant is a block POE-POP polymer.

9. The composition of claim 5, wherein said positive contrast agent is a paramagnetic metal chelate which, after administration, readily distributes extracellularly.

10. The composition of claim 9, wherein the paramagnetic chelate is Gd-DTPA, Gd-BOPTA, Gd-DOTA, Mn-EDTA or Gd-DO3A.

11. The composition of claim 6, wherein said non-ionic surfactant is a block POEG-POEP polymer, a polyoxyethylene fatty acid ester, a polyoxyethylene sorbitan fatty acid ester, an n-alkylglucopyranoside, or an n-alkyl maltotrioside.

12. The composition of claim 6, wherein said phosphorous compound is a phospholipid and is selected from phosphatidic acid, phosphatidylcholine, phosphatidylethanolamine, phosphatidylserine, phosphatidylglycerol, phosphatidyl-inositol, cardiolipin and sphingomyelin.

13. The composition of claim 12, wherein said phospholipid consists of a mono-phosphate ester of a substituted or partially substituted glycerol, at least one functional group of said glycerol being esterified by saturated or unsaturated aliphatic fatty acid, or etherified by saturated or unsaturated alcohol, the other two acidic functions of the phosphoric acid being either free or salified with alkali or earth-alkali metals.

14. The composition of claim 3, wherein said lipophilic moiety of the paramagnetic metal complex is a C₁ to C₂₄ alkyl or alkylene group or a substituted or unsubstituted benzyl- or phenyl-alkyl group.

15. The composition of claim 14, wherein said lipophilic moiety of the paramagnetic metal complex is a carboxylate ester of saturated and unsaturated C₁ to C₂₄ aliphatic or aromatic alcohols or is a carboxylate amide of saturated and unsaturated C₁ to C₂₄ aliphatic or aromatic amines.

16. The composition of claim 2, wherein said magnetite particles of the negative contrast agent are nanometric in size and are optionally dextran coated.

17. A kit comprising a positive contrast agent component acting on the T₁ proton relaxation factor, and a negative contrast agent component acting on the T₂ proton relaxation factor.

18. The kit of claim 17, wherein the positive agent is a blood remnant paramagnetic metal chelate compound, and the other contrast component comprises submicronic magnetite particles.

19. The kit of claim 17, wherein the negative agent is a blood remnant compound, and the other contrast component comprises a paramagnetic chelate.

20. The kit of claim 18 or 19, wherein at least one of the components is in the form of a dry powder.

21. The kit of claim 18 or 19, wherein both components are in the form of a dry powder.

22. The kit of claim 20 or 21, wherein the kit further comprises a vial containing physiologically acceptable carrier liquid which when admixed with dry powdery formulation provides, an injectable MRI contrast composition of claims 1-16.

23. A method of making dual MRI contrast medium of claims 1-16.

24. An MRI method for improved visualization of the circulation against its surroundings in organs of live subjects, comprising administering to said subjects a dual contrast composition according to claim 1, and MRI monitoring at intervals the organs of interest, whereby enhanced contrast between the circulation and the surroundings is obtained.
